(19) United States
(12) Patent Application Publication
(10) Pub. No.: US 2010/0216958 A1
(43) Pub. Date: Aug. 26, 2010

(54) Methods of Preparing Renewable Butadiene and Renewable Isoprene

(76) Inventors: Matthew W. Peters, Highlands Ranch, CO (US); Joshua D. Taylor, Evergreen, CO (US); Leo E. Manzer, Wilmington, DE (US); David E. Henton, Midland, MI (US)

Correspondence Address: COOLEY LLP;ATTN: Patent Group, Suite 1100, 777 - 6th Street, NW, WASHINGTON, DC, 20001, US

(21) Appl. No.: 12/711,919

(22) Filed: Feb. 24, 2010

Related U.S. Application Data
(60) Provisional application No. 61/155,029, filed on Feb. 24, 2009.

(51) Int. Cl. C07C 5/09; C08F 226/06; C08F 10/00; C08F 134/00; C08F 12/28; C08F 120/18; C08F 136/00; C08F 220/44; C08F 212/08; C08F 210/04; C08G 18/00; C08G 63/02; C08G 69/16; C08G 65/34; C07D 201/02; C07D 295/02; C07C 55/14; C07C 211/22; C07C 29/03; C07C 17/02; C07C 5/333; C07C 4/00; C07C 1/20; C07C 5/00

(52) U.S. Cl. 526/258; 526/284; 526/295; 526/310; 526/329.7; 526/335; 526/342; 526/347; 526/348.6; 526/348.7; 528/44; 528/272; 528/323; 528/425; 540/538; 548/579; 562/590; 564/511; 568/860; 570/216; 585/16; 585/318; 585/327; 585/500; 585/627

(57) ABSTRACT

Isobutene, isoprene, and butadiene are obtained from mixtures of C₄ and/or C₅ olefins by dehydrogenation. The C₄ and/or C₅ olefins can be obtained by dehydration of C₄ and C₅ alcohols, for example, renewable C₄ and C₅ alcohols prepared from biomass by thermochemical or fermentation processes. Isoprene or butadiene can be polymerized to form polymers such as polyisoprene, polybutadiene, synthetic rubbers such as butyl rubber, etc. in addition, butadiene can be converted to monomers such as methyl methacrylate, adipic acid, adiponitrile, 1,4-butadiene, etc. which can then be polymerized to form nylons, polyesters, polymethylmethacrylate etc.

### CROSS REFERENCE TO RELATED APPLICATIONS

The present application claims priority to U.S. Provisional Application No. 61/155,029 filed Feb. 24, 2009, which is incorporated herein by reference in its entirety for all purposes.

### BACKGROUND OF THE INVENTION

Butadiene and isoprene are important industrial chemicals typically used as monomers for producing a variety of synthetic polymers, including synthetic rubber. Butadiene is conventionally produced as a byproduct of steam cracking processes (used in petroleum refining to produce ethylene and other olefins). Steam cracking typically produces a complex mixture of unsaturated hydrocarbon, including butadiene, and the amount of butadiene produced depends upon the particular petroleum feedstock used, as well as the operating conditions employed. Butadiene is typically removed from the resulting relatively complex mixture of hydrocarbons by extraction into a polar aprotic solvent (such as acetonitrile or dimethylformamide), from which it is then stripped by distillation. Butadiene can also be produced by the catalytic dehydrogenation of n-butane and n-butenes (n-butane is also produced as part of a complex mixture of light hydrocarbons in petroleum refining processes).

Isoprene is also produced during petroleum refining, typically as a byproduct of a thermal cracking process, or as a byproduct in the production of ethylene (typically 2-5% of the ethylene yield). Additionally, isoprene can be prepared from isobutene via a combined hydroformylation and dehydration process (e.g., as described in U.S. Pat. No. 3,662,016), or via condensation with formaldehyde (e.g. Prins condensation; see FIG. 1). However, the C₅ hydrocarbons produced by cracking operations generally contain large amounts of cyclopentadiene, which has a similar boiling point to isoprene. Accordingly, isoprene is difficult to separate from cyclopentadiene using conventional distillation methods. Alternative techniques are often used, such as first thermally dimerizing the cyclopentadiene component before distilling, or extractively distilling the isoprene with polar solvents.

Butadiene and isoprene are major components of commercially useful polymers (e.g., rubbers and elastomers). However, polymerization catalysts used to prepare such materials are typically intolerant of impurities, and therefore require relatively pure butadiene and isoprene (and other monomers). Because petrochemically derived butadiene and isoprene are obtained from complex hydrocarbon mixtures, it is usually necessary to carry out extensive (and expensive) purification prior to polymerization. Accordingly, processes capable of directly providing relatively pure butadiene or isoprene which require little or no additional purification would be desirable.

Furthermore, there is increasing concern that the use of petroleum-derived hydrocarbons as basic raw materials (e.g., butadiene or isoprene) contributes to environmental degradation (e.g., global warming, air and water pollution, etc.) and fosters overdependence on unreliable petroleum supplies from politically unstable parts of the world. Accordingly, it would be desirable to obtain renewable (i.e., biologically derived) sources of industrially important monomers such as butadiene and isoprene.

The present invention is directed to improved methods for preparing butadiene and isoprene, particularly renewable butadiene and isoprene, which are simple, economical, do not require difficult and expensive extraction of starting materials from fermentation broths, or extensive purification of the butadiene or isoprene. Butadiene and isoprene prepared by the methods of the present invention are suitable for preparing renewable polymers, copolymers, and other materials derived therefrom.

### SUMMARY OF THE INVENTION

In one embodiment, the present invention is directed to a method of preparing butadiene comprising (a) providing an alcohol mixture comprising one or more butanols; (b) contacting the alcohol mixture with a dehydration catalyst, thereby forming an olefin mixture comprising one or more linear butenes and isobutene; (c) contacting the olefin mixture of step (b) with a dehydrogenation catalyst, thereby forming a di-olefin mixture comprising butadiene and isobutene; and (d) isolating butadiene from the di-olefin mixture of (c).

In another embodiment, the present invention is directed to a method of preparing isoprene comprising (a) providing an olefin mixture comprising one or more pentenes, with the proviso that at least a portion of the olefin mixture comprises one or more methylbutenes; (b) contacting the olefin mixture of (a) with a dehydrogenation catalyst, thereby forming a mixture comprising isoprene; and (c) isolating isoprene from the mixture of (b).

In still another embodiment, the present invention is directed to a method of preparing monomers, comprising: (a) providing an olefin mixture comprising one or more linear butenes and isobutene; (b) contacting the olefin mixture of step (a) with a dehydrogenation catalyst, thereby forming a di-olefin mixture comprising butadiene and isobutene; (c) isolating isobutene from the mixture of step (b); and (d1) converting the isobutene to methyl t-butyl ether, ethyl t-butyl ether, isooctane, methacrolein, methyl methacrylate, butyl rubber, butylated hydroxytoluene, or butylated hydroxyanisole.

In still other embodiments, the present invention is directed to methods for preparing isobutene or isoprene as described herein, wherein the olefin mixture is prepared by dehydration of a renewable alcohol mixture comprising one or more renewable C₄ or C₅ alcohols.

In still further embodiments, the present invention is directed to renewable isobutene, renewable isoprene, renewable butadiene, renewable methyl methacrylate, renewable 1,4-butanediol, renewable THF, renewable N-vinylpyrrolidinone, renewable lauryllactam, renewable chloroprene, renewable adipic acid, renewable hexamethylenediamine, renewable caprolactam, and renewable ethylidene norbornene, as well as renewable polymers and copolymers prepared from these renewable monomers.

In yet another embodiment, the present invention is directed to a method of preparing isobutene, comprising (a) providing an olefin mixture comprising one or more linear butenes and isobutene; (b) contacting the olefin mixture of (a) with a dehydrogenation catalyst, thereby forming a di-olefin mixture comprising butadiene and isobutene; and (c) isolating high purity isobutene from the mixture of (b).

### BRIEF DESCRIPTION OF THE DRAWINGS

FIG. 1: Schematic of preparing isoprene by the Prins reaction.

FIG. 2: Schematic of isobutanol dehydration.

FIG. 3: Schematic of one embodiment of a dehydration reactor configuration.

FIG. 4: Equilibrium concentration of various C₄-olefins as a function of temperature.

FIG. 5: Schematic of dehydrogenation of n-butane to 1- and 2-butenes.

FIG. 6: Schematic of dehydrogenation of 1-butene to 1,3-butadiene.

FIG. 7: Schematic of skeletal rearrangement of isobutene.

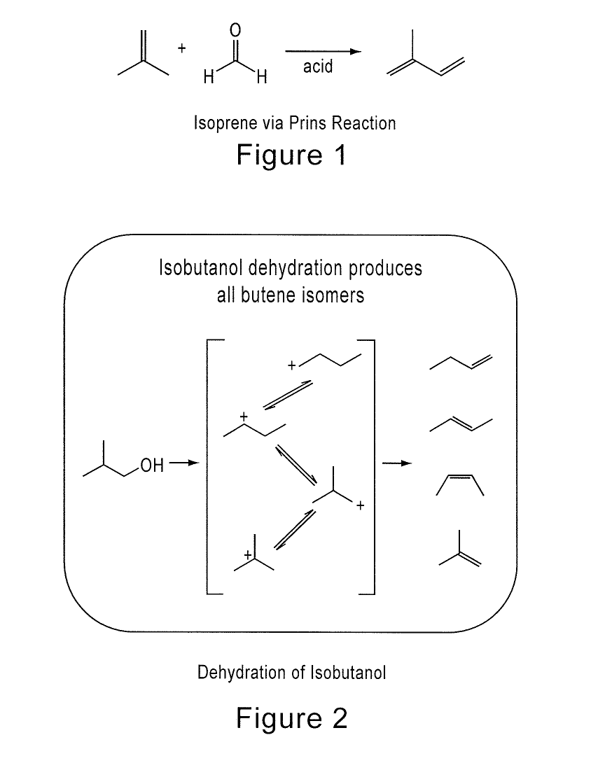

### DETAILED DESCRIPTION OF THE INVENTION

All documents cited herein are incorporated by reference in their entirety for all purposes to the same extent as if each individual document was specifically and individually indicated to be incorporated by reference.

In conventional petrochemical processes for preparing butadiene, butadiene is a coproduct produced during the steam cracking of naphtha and gas-oil fractions, or produced by catalytic dehydrogenation of n-butane or n-butene (which themselves are obtained by steam cracking). The crude 1,3-butadiene-containing fraction includes various C₃-C₅ hydrocarbons, including propylene, propane, isobutylene, 1-butene, n-butane, trans-2-butene, cis-2-butene, C₄ acetylenes, 1,2-butadiene, various C₅ hydrocarbons, etc., depending upon the particulars of the process and conditions. For use as a monomer in preparing polymers (e.g. synthetic rubber), butadiene must be relatively pure (e.g. at least about 99.0 wt. %) in order to prevent deactivation of conventional polymerization catalysts, or to prevent side reactions due to reactive impurities (such as acetylenes). Various methods for purifying crude butadiene produce from it for chemical sources have been used, for example selective extraction with aqueous sucrose ammonium acetate or extractive distillation with various solvents. The need for such purification methods add additional expense and complexity in preparing polymerization-grade butadiene.

Similarly, isoprene is typically obtain from C₅ streams from thermally cracking naphtha and gas oil. Yields of isoprene are generally small, and isoprene, like butadiene, must be purified from quite complex mixtures of hydrocarbons before it can be used as a monomer.

Various methods for preparing renewable 1,3-butadiene from butane diols have been proposed: fermentation of sugars to 2,3-butanediol, which is then dehydrated to 1,3-butadiene (e.g. U.S. Pat. No. 2,529,061 and Syu M J et al Applied Microbiology and Biotechnology 2001, 55, 10-18); fermentation of sugars to 1,4-butanediol, and subsequent dehydration to 1,3-butadiene (e.g. press release by Genomatica, Inc.); and fermentation of sugars to succinate, hydrogenation of the succinate to 1,4-butanediol, then dehydration of the 1,4-butanediol to 1,3-butadiene (e.g. Delhomme C et al Green Chemistry 2009, 11, 13-26). However, commercial-scale production of butadiene by these routes is generally considered too difficult and costly because of the known difficulty (and consequent expense) of removing diols and diacids from a fermentation broth.

The methods of the present invention provide an improved process for preparing butadiene (or isoprene) by sequential dehydration and dehydrogenation reactions from a relatively pure butanol (or pentanol) feedstock, for example isobutanol (or 3-methyl-1-butanol). As described herein, the dehydration step provides a relatively simple mixture of butene isomers which can be converted directly to butadiene by dehydrogenation. Any byproduct of the dehydration which cannot be converted directly to butadiene (or isoprene) can be readily removed, either from the mixture of linear butene isomers (or methylbutene isomers), or from the butadiene (or isoprene) of the product stream of the dehydrogenation step. Yields of butadiene (or isoprene) can be further increased by appropriate conversion of these byproducts (e.g. recycling and/or rearrangement as described herein), or the byproducts can be used for other purposes (e.g., as fuels or fuel additives). Thus, the present invention provides a simple process for obtaining relatively pure butadiene from butanols (or isoprene from pentanols). Furthermore, if the butanols (or pentanols) are derived from biomass (e.g., by fermentation of biomass-derived carbohydrates using suitable microorganisms), the butanols (or pentanols) are obtained as a relatively pure (usually aqueous) feedstock. Biomass derived butanols (or pentanols) have the additional advantage of providing a renewable source of a commercially important monomer, butadiene (or isoprene). In addition, it was unexpectedly found that olefins prepared by dehydration from biomass derived butanols (or pentanols), as described herein, are substantially purer than, e.g., butenes or pentenes obtained from conventional petrochemical processes (e.g., obtained by “cracking”).

“Renewably-based” or “renewable” denote that the carbon content of the renewable alcohol (and olefin, di-olefin, etc., or subsequent products prepared from renewable alcohols, olefins, di-olefins, etc. as described herein), is from a “new carbon” source as measured by ASTM test method D 6866-05, “Determining the Biobased Content of Natural Range Materials Using Radiocarbon and Isotope Ratio Mass Spectrometry Analysis”, incorporated herein by reference in its entirety. This test method measures the ¹⁴C/¹²C isotope ratio in a sample and compares it to the ¹⁴C/¹²C isotope ratio in a standard 100% biobased material to give percent biobased content of the sample. “Biobased materials” are organic materials in which the carbon comes from recently (on a human time scale) fixated CO₂ present in the atmosphere using sunlight energy (photosynthesis). On land, this CO₂ is captured or fixated by plant life (e.g., agricultural crops or forestry materials). In the oceans, the CO₂ is captured or fixated by photosynthesizing bacteria or phytoplankton. For example, a biobased material has a ¹⁴C/¹²C isotope ratio greater than 0. Contrarily, a fossil-based material, has a ¹⁴C/¹²C isotope ratio of about 0. The term “renewable” with regard to compounds such as alcohols or hydrocarbons (olefins, di-olefins, polymers, etc.) also refers to compounds prepared from biomass using thermochemical methods (e.g., Fischer-Tropsch catalysts), biocatalysts (e.g., fermentation), or other processes, for example as described herein.

A small amount of the carbon atoms of the carbon dioxide in the atmosphere is the radioactive isotope ¹⁴C. This ¹⁴C carbon dioxide is created when atmospheric nitrogen is struck by a cosmic ray generated neutron, causing the nitrogen to lose a proton and form carbon of atomic mass 14 (¹⁴C), which is then immediately oxidized to carbon dioxide. A small but measurable fraction of atmospheric carbon is present in the form of ¹⁴CO₂. Atmospheric carbon dioxide is processed by green plants to make organic molecules during the process known as photosynthesis. Virtually all forms of life on Earth depend on this green plant production of organic molecules to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from fossil fuels does not have the signature ¹⁴C:¹²C ratio of renewable organic molecules derived from atmospheric carbon dioxide. Furthermore, renewable organic molecules that biodegrade to CO₂ do not contribute to global warming as there is no net increase of carbon emitted to the atmosphere.

Assessment of the renewably based carbon content of a material can be performed through standard test methods, e.g. using radiocarbon and isotope ratio mass spectrometry analysis. ASTM International (formally known as the American Society for Testing and Materials) has established a standard method for assessing the biobased content of materials. The ASTM method is designated ASTM-D6866.

The application of ASTM-D6866 to derive “biobased content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modern carbon). If the material being analyzed is a mixture of present day radiocarbon and fossil carbon (containing very low levels of radiocarbon), then the pMC value obtained correlates directly to the amount of biomass material present in the sample.

Throughout the present specification, reference to alcohols, olefins, di-olefins, etc., and higher molecular weight materials (e.g., isooctene/isooctane, polymers, copolymers, etc.) made from such compounds is synonymous with “renewable” alcohols, “renewable” olefins, “renewable” di-olefins, etc., and “renewable” materials (e.g., “renewable” isooctene/isooctane, “renewable” polymers, “renewable” copolymers, etc.) unless otherwise indicated.

Throughout the present specification, the term “butadiene” refers to 1,3-butadiene unless otherwise indicated.

As described herein, the methods of the present invention can be used to prepare butadiene, isoprene, isobutene, etc. suitable for use in polymerization reactions or other processes which require relatively high purity. The term “high purity” means at least about 95% pure, at least about 96% pure, at least about 97% pure, at least about 98% pure, at least about 99% pure, at least about 99.9% pure, or at least about 99.99% pure, including all ranges and subranges therebetween.

The renewable alcohols, olefins, di-olefins, polymers, etc. of the present invention have pMC values of at least about 1, 5, 10, 15, 20, 25, 30, 35, 40, 45, 50, 55, 60, 65, 70, 75, 80, 85, 90, 95, 100, inclusive of all values and subranges therebetween.

Any suitable microorganism can be used to prepare renewable butanols and pentanols. Butanols are preferentially produced, for example, by the microorganisms described in U.S. Patent Publication Nos. 2007/0092957, 2008/0138870, 2008/0182308, 2007/0259410, 2007/0292927, 2007/0259411, 2008/0124774, 2008/0261230, 2009/0226991, 2009/0226990, 2009/0171129, 2009/0215137, 2009/0155869, 2009/0155869, 2008/02745425, etc. Additionally, butanols and isobutanols and various pentanols including isopentanol are produced by yeasts during the fermentation of sugars into ethanol. These fusel alcohols are known in the art of industrial fermentations for the production of beer and wine and have been studied extensively for their effect on the taste and stability of these products. Recently, production of fusel alcohols using engineered microorganisms has been reported (U.S. Patent Application No. 2007/0092957, and Nature, 2008, 451, p. 86-89).

Higher alcohols other than butanols or pentanols produced during fermentation (or other processes as described herein for preparing renewable butanols or pentanols) may be removed from the butanol or pentanol feedstocks prior to carrying out the subsequent unit operations (e.g., dehydration). The separation of these higher alcohols from the butanol(s) (e.g. isobutanol) or pentanol(s) (e.g. isopentanol) can be effected using known methods such as distillation, extraction, etc. Alternatively, these higher alcohols can remain mixed in the butanol(s) or pentanol(s), and removed after subsequent processing. For example, any higher alcohols mixed in with isobutanol can be dehydrated to the corresponding olefins, which can then be separated from the butenes. The determination of whether to remove such higher alcohols prior to dehydration, or to remove the corresponding olefin after dehydration (or the corresponding dehydrogenation byproducts/co-products) will depend on the relative ease of respective separations, and the relative value of the byproducts/co-products.

Renewable butanols or pentanols can also be prepared using various other methods such as conversion of biomass by thermochemical methods, for example by gasification of biomass to synthesis gas followed by catalytic conversion of the synthesis gas to alcohols in the presence of a catalyst containing elements such as copper, aluminum, chromium, manganese, iron, cobalt, or other metals and alkali metals such as lithium, sodium, and/or potassium (Energy and Fuels, 2008, 22, p. 814-839). The various alcohols, including butanols and pentanols can be separated from the mixture by distillation and used to prepare renewable butadiene or isoprene, or compounds derived from renewable butadiene or isoprene as described herein. Alcohols other than isobutanol and isopentanol can be recovered and utilized as feedstocks for other processes, burned as fuel or used as a fuel additive, etc.

Alternatively, renewable alcohols can be prepared photosynthetically, e.g., using cyanobacteria or algae engineered to produce isobutanol, isopentanol, and/or other alcohols (e.g., Synechococcus elongatus PCC7942 and Synechocystis PCC6803; see Angermayr et al., Energy Biotechnology with Cyanobacteria, Current Opinion in Biotechnology 2009, 20, 257-263, Atsumi and Liao, Nature Biotechnology, 2009, 27, 1177-1182); and Dexter et al., Energy Environ. Sci., 2009, 2, 857-864, and references cited in each of these references). When produced photosynthetically, the “feedstock” for producing the resulting renewable alcohols is light and the CO₂ provided to the photosynthetic organism (e.g., cyanobacteria or algae).

Renewable and pure butanols and pentanols obtained by biochemical or thermochemical production routes can be converted into their corresponding olefins by reacting the alcohols over a dehydration catalyst. Renewable butanols typically comprise 1-butanol, 2-butanol, or isobutanol, but tent-butanol may also be obtained by thermochemical routes. Renewable pentanols typically comprise 1-pentanol, 2-methyl-1-butanol, and 3-methyl-1-butanol, but most pentanol isomers are produced by thermochemical and, less commonly, by fermentation routes.

When the renewable butanols (e.g., isobutanol) or pentanols (e.g., 3-methyl-1-butanol) are prepared by fermentation, the isobutanol can be removed from the fermentor by various methods, for example in the vapor phase under reduced pressure (e.g. as an azeotrope with water as described in US 2009/0171129). In some such embodiments, the fermentor itself is operated under reduced pressure without the application of additional heat (other than that used to provide optimal fermentation conditions for the microorganism) or the use of distillation equipment, whereby the isobutanol is removed as an aqueous vapor (or azeotrope). In other such embodiments, the fermentor is operated under approximately atmospheric pressure (or slightly elevated pressure due to the evolution of gases such as CO₂ during fermentation) and a portion of the feedstock containing the isobutanol is continuously recycled through a flash tank operated under reduced pressure, whereby the isobutanol is removed from the headspace of the flash tank as an aqueous vapor or water azeotrope. These latter embodiments have the advantage of providing for separation of the isobutanol without the use of energy intensive or equipment intensive unit operations, as well as continuously removing a metabolic by-product of the fermentation and thereby improving the productivity of the fermentation process. The resulting wet isobutanol can be dried and then dehydrated, or dehydrated wet (as described herein), then subsequently dried.

The production of renewable isobutanol by fermentation of carbohydrates co-produces small (<5% w/w) amounts of 3-methyl-1-butanol and 2-methyl-1-butanol and much lower levels of other fusel alcohols. One mechanism by which these by-products form is the use of intermediates in hydrophobic amino acid biosynthesis by the isobutanol-producing metabolic pathway that is engineered into the host microorganism. The genes involved with the production of intermediates that are converted to 3-methyl-1-butanol and 2-methyl-1-butanol are known and can be manipulated to control the amount of 3-methyl-1-butanol produced in these fermentations (e.g., Connor M R and Liao J C, Applied and Environmental Microbiology 2008, 74, p. 5769). Removal of these genes can decrease 3-methyl-1-butanol and/or 2-methyl-1-butanol production to negligible amounts, while overexpression of these genes can be tuned to produce any amount of 3-methyl-1-butanol in a typical fermentation. Alternatively, the thermochemical conversion of biomass to mixed alcohols produces both isobutanol and these pentanols. The relative amounts of these alcohols can be tuned using specific catalysts and reaction conditions.

Alcohols can be converted to olefins by reaction with a suitable dehydration catalyst under appropriate conditions (see e.g., FIG. 2). Typical dehydration catalysts that convert alcohols such as butanols and pentanols into olefins include various acid treated and untreated alumina (e.g., γ-alumina) and silica catalysts and clays including zeolites (e.g., β-type zeolites, ZSM-5 or Y-type zeolites, fluoride-treated β-zeolite catalysts, fluoride-treated clay catalysts, etc.), sulfonic acid resins (e.g., sulfonated styrenic resins such as Amberlyst® 15), strong acids such as phosphoric acid and sulfuric acid, Lewis acids such boron trifluoride and aluminum trichloride, and many different types of metal salts including metal oxides (e.g., zirconium oxide or titanium dioxide) and metal chlorides (e.g., Latshaw B E, Dehydration of Isobutanol to Isobutylene in a Slurry Reactor, Department of Energy Topical Report, February 1994).

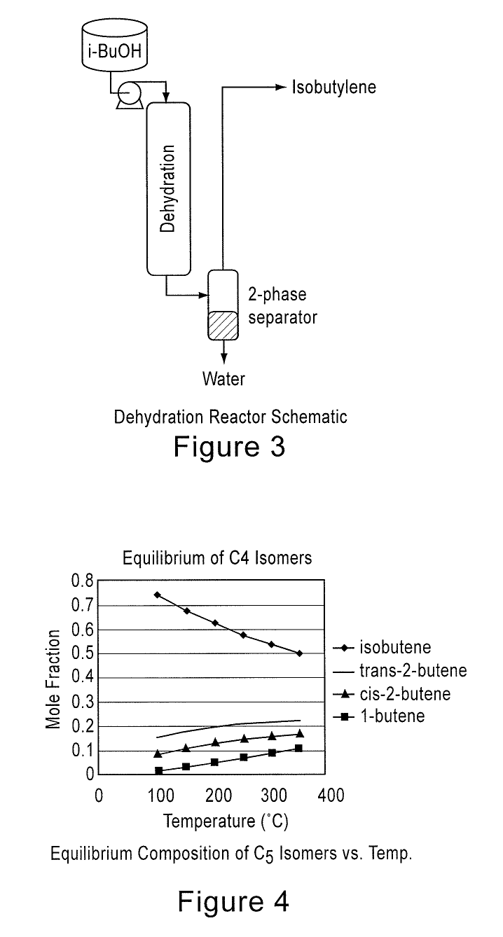

Dehydration reactions can be carried out in both gas and liquid phases with both heterogeneous and homogeneous catalyst systems in many different reactor configurations (see e.g. FIG. 3). Typically, the catalysts used are stable to the water that is generated by the reaction. The water is usually removed from the reaction zone with the product. The resulting alkene(s) either exit the reactor in the gas or liquid phase (e.g., depending upon the reactor conditions) and are captured by a downstream purification process or are further converted in the reactor to other compounds (such as butadiene or isoprene) as described herein. The water generated by the dehydration reaction exits the reactor with unreacted alcohol and alkene product(s) and is separated by distillation or phase separation. Because water is generated in large quantities in the dehydration step, the dehydration catalysts used are generally tolerant to water and a process for removing the water from substrate and product may be part of any process that contains a dehydration step. For this reason, it is possible to use wet (i.e., up to about 95% or 98% water by weight) alcohol as a substrate for a dehydration reaction and remove this water with the water generated by the dehydration reaction (e.g., using a zeolite catalyst as described U.S. Pat. Nos. 4,698,452 and 4,873,392). Additionally, neutral alumina and zeolites will dehydrate alcohols to alkenes but generally at higher temperatures and pressures than the acidic versions of these catalysts.

When 1-butanol, 2-butanol, or isobutanol are dehydrated, a mixture of four C₄ olefins—1-butene, cis-2-butene, trans-2-butene, and isobutene—is formed. The exact concentration of each olefin is determined by the starting material, by thermodynamics (FIG. 4), and by the reaction conditions and catalysts used. It is possible to understand how these factors affect the distribution of olefins in the final product and use this knowledge to obtain mixtures enriched in a particular olefin. However, production of a single olefin by the dehydration of one of these alcohols is generally difficult. For example, dehydration of isobutanol at 280° C. over a γ-alumina catalyst can be optimized to produce up to 97% isobutene despite an expected equilibrium concentration of ˜57% at that temperature (FIG. 3). However, there is no known method for cleanly dehydrating isobutanol to 99+% isobutene (Saad L and Riad M, Journal of the Serbian Chemical Society 2008, 73, p. 997).

Similarly, dehydration of pentanols produces multiple C₅-olefin isomers. For example, dehydration of 3-methyl-1-butanol produces both 3-methyl-1-butene and 2-methyl-2-butene in addition to other olefin isomers (see e.g. US 2007/0135665 A1). Dehydration of 2-methyl-1-butanol will produce primarily 2-methyl-1-butene and 2-methyl-2-butene but some skeletal rearrangement will occur to produce linear 1-pentene and 2-pentene. Dehydrogenation of these pentene mixtures produce isoprene and linear pentadienes that are fairly easy to separate to produce pure isoprene.

As discussed above, di-olefins such as butadiene and isoprene are conventionally produced in the cracking reactions that generate C₄ and C₅ olefin streams for petrochemical use. If additional di-olefins are required, they can be produced by dehydrogenation of C₄ and C₅ mono-olefins. For example, butadiene is produced by passing raffinate-2 over a dehydrogenation catalyst. Isoprene is similarly produced by passing isopentane and/or 3-methyl-1-butene and 2-methyl-2-butene over a dehydrogenation catalyst. Alternatively, isoprene can be produced by the hydroformylation and dehydration of isobutene.

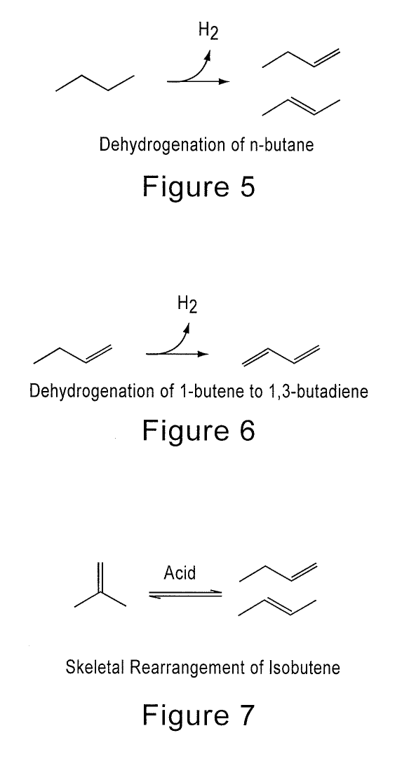

Dehydrogenation catalysts convert saturated carbon-carbon bonds in organic molecules into unsaturated double bonds (see FIG. 5). Typical dehydrogenation catalysts are mixtures of metal oxides with varying degrees of selectivity towards specific olefins. For example, iron-zinc oxide mixtures appear to favor 1-butene dehydrogenation while cobalt-iron-bismuth-molybdenum oxide mixtures favor 2-butene dehydrogenation (e.g., Jung J C, et al., Catalysis Letters 2008, 123, p. 239). Other examples of dehydrogenation catalysts include vanadium- and chrome-containing catalysts (e.g., Toledo-Antonio J A, et al., Applied Catalysis A 2002, 234, p. 137), ferrite-type catalysts (e.g., Lopez Nieto J M, et al., Journal of Catalysis 2000, 189, p. 147), manganese-oxide doped molecular sieves (e.g., Krishnan V V and Suib S L, Journal of Catalysis 1999, 184, p. 305), copper-molybdenum catalysts (e.g., Tiwari P N, et al., Journal of Catalysis 1989, 120, p. 278), and bismuth-molybdenum-based catalysts (e.g., Batist P A, et al., Journal of Catalysis 1966, 5, p. 55).

Dehydrogenation of an olefin to a di-olefin occurs if the olefin molecule can accommodate an additional double bond (see FIG. 6). For example, 1-butene can be dehydrogenated to butadiene but isobutene cannot be dehydrogenated unless skeletal rearrangement of the carbon atoms in the molecule occurs. Dehydrogenation catalysts are capable of rearranging olefinic bonds in a molecule to accommodate a second olefin bond if skeletal rearrangement is not required (e.g., by one or more hydrogen shifts), but these catalysts typically do not catalyze skeletal rearrangements (e.g., breaking and reforming C—C bonds) under dehydrogenating conditions. For example, 2-butene can be dehydrogenated to butadiene. Similarly, 2-methyl-2-butene can be converted to isoprene after rearrangement of the double bond.

Two major types of dehydrogenation reactions are conventionally used to produce olefins from saturated materials (Buyanov R A, Kinetics and Catalysis 2001, 42, p. 64). Endothermic dehydrogenation uses a dehydrogenation catalyst (e.g. chromia-alumina-based, spinel supported platinum-based, phosphate-based, and iron oxide-based catalysts), high heat (typically 480-700° C.), and a reactor configuration (typically fixed-bed and fluidized-bed reactors) that favors the formation of hydrogen gas to drive the reaction forward, and by diluting the feedstock with gases such as helium, nitrogen, hydrogen, or steam to lower the partial pressure of any hydrogen that is formed in the reaction or placing the reaction under reduced pressure (0.1 to 0.7 atm). Endothermic dehydrogenation catalysts typically function in the absence of oxygen, minimizing the formation of oxidized butene products such as methacrolein and methacrylate. Oxidative dehydrogenation typically uses mixed metal oxide-based dehydrogenation catalyst (typically containing molybdenum, vanadium, or chromium), lower temperatures (300-500° C.), and a fixed- or fluidized-bed reactor configuration that includes the addition of oxygen to the reaction to drive the reaction by reacting with hydrogen to form water. Both types of dehydrogenation reactions are applicable to the invention described herein.

As discussed above, dehydration of butanols and pentanols usually produces a mixture of mono-olefins (e.g., linear butenes and isobutylene, or various pentenes). Thus, for example, the dehydration of isobutanol generally produces a mixture of linear butenes (1-butene and 2-butenes) and isobutene. As discussed herein, linear butenes are readily dehydrogenated to butadiene, whereas under typical dehydrogenation conditions, isobutene is relatively inert. Accordingly, in some embodiments, it may be desirable to remove isobutene from the dehydration product/dehydrogenation feedstock. Alternatively, the mixture of linear butenes and isobutene can be dehydrogenated to produce a dehydrogenation product stream comprising butadiene, unreacted isobutene, and optionally unreacted linear butenes. In most embodiments, the linear butenes would be recycled back to the dehydrogenation reactor to further convert the linear butenes to butadiene (thereby increasing the effective yield of butadiene). The unreacted isobutene can be readily separated from butadiene, and recycled to a separate rearrangement step (i.e., producing linear butenes as shown in FIG. 7) or diverted to other processes (e.g., oligomerization, oxidation, etc. to produce biofuels, acrylates, aromatics, etc.) as described herein. If the unreacted isobutene is rearranged to linear butenes, the linear butenes can be recycled back to the dehydrogenation step to produce additional butadiene.

In still other embodiments, the mixed butenes can be oligomerized over an acidic ion exchange resin under conditions which selectively convert isobutene to isooctene (e.g. using the methods of Kamath R S et al, Industrial Engineering and Chemistry Research 2006, 45, 1575-1582), but leave the linear butenes essential unreacted, thereby providing an essentially isobutene-free mixture of linear butenes (containing e.g., less than about 1% isobutene). The essentially isobutene-free renewable linear butenes can then be reacted in the presence of a dehydrogenation catalyst to form renewable butadiene.

The selectivity of dehydrogenation catalysts towards olefins that can accommodate a second olefinic bond can be used to prepare butadiene or isoprene, or alternatively purify the olefin mixture (e.g. by facilitating separation of the diene from unreactive mono-olefins). For example, as described herein, the dehydration of isobutanol typically produces isobutene and both 1- and 2-butenes. Treatment of this product mixture with a dehydrogenation catalyst selectively converts the 1- and 2-butenes—but not isobutene—to butadiene. It is possible that some skeletal rearrangement of the isobutene occurs during the dehydrogenation reaction, but this rearranged material dehydrogenates to form butadiene. After complete dehydrogenation (which may require recycling unreacted butenes back to the dehydrogenation feedstock), the butadiene and unreacted isobutene are readily separated by extractive distillation of the butadiene, to produce high purity (about 80-100%, e.g., >about 80%, >about 85%, >about 90%, >about 95%, >about 98%, >about 99%, or >about 99.8%) isobutene and butadiene suitable e.g. for use as a monomer feedstock for polymerization.

In another embodiment, 1- and 2-butanol are dehydrated to produce mixtures of butenes that are primarily comprised of linear butenes with small amounts (<15% w/w) of isobutene. The isobutene can be separated from these mixtures by dehydrogenation using a method similar to that described above, especially if butadiene is the desired product. If isobutanol is the only available feedstock and butadiene is a desired product, the amount of 1- and 2-butenes produced in the dehydration of isobutanol can be increased up to the equilibrium amount accessible at the reaction temperature (see e.g. FIG. 3). For example, in some embodiments, dehydration catalysts are selected such that at 350° C. the dehydration of isobutanol produces 50% isobutene and 50% 1- and 2-butenes. The resulting mixture is treated with a dehydrogenation catalyst to produce butadiene from isobutanol at a 50% yield.

In various embodiments the isobutene can be removed from the mixture of linear butenes prior to dehydrogenation, or alternatively, if the dehydrogenation conditions and catalyst are selected to minimize any undesired side reactions of the isobutene, the isobutene can removed from the product stream after the dehydrogenation reaction step. In other embodiments, a portion or all of the isobutene can be diverted to form other valuable hydrocarbons (e.g., oligomerized to form isooctenes/isoctanes for biofuels, dehydrocyclized to form aromatics for fuels, phthalates, etc.). The isobutene can also be rearranged to linear butenes (1- and 2-butenes), which can then be recycled back to the dehydrogenation reaction step to form additional butadiene, thereby increasing the effective yield of butadiene well above 50%. If all of the isobutene is recycled, the effective yield of butadiene in various processes of the present invention can approach about 100%. However, as some cracking and “coking” may occur during the dehydrogenation, butadiene yields for the process of the present invention can be about 90% or more, about 95% or more, or about 98% or more. The rearrangement of isobutene can be carried out in a separate isomerization step (e.g., in a separate isomerization reactor) after removing the butadiene from the dehydrogenation product, or can be carried out in-situ during the dehydrogenation reaction by appropriate selection of catalyst (or by use of a catalyst mixture) in the dehydrogenation reactor. For example, dehydration catalysts can be selected which also catalyze rearrangement of isobutene to linear isobutenes, or the dehydration catalyst can be mixed with an isomerization catalyst. A few representative acid catalysts suitable for rearranging isobutene include zeolites such as CBV-3020, ZSM-5, β Zeolite CP 814C, ZSM-5 CBV 8014, ZSM-5 CBV 5524 G, and YCBV 870; fluorinated alumina; acid-treated silica; acid-treated silica-alumina; acid-treated titania; acid-treated zirconia; heteropolyacids supported on zirconia, titania, alumina, silica; and combinations thereof.

In particular embodiments, the isobutene is substantially removed from the product stream after the dehydration reaction step in order to provide a feed stream for the dehydrogenation reaction step which is substantially free of isobutene (i.e., the butene component of the dehydrogenation feed stream comprises substantially only linear butenes). By “substantially removed” we mean that isobutene has been removed from the indicated feed or product stream such that after removal, the isobutene in the feed or product stream comprises less than about 5%, less than about 4%, less than about 3%, less than about 2%, or less than about 1% of the butenes in the indicated feed or product stream. By “substantially only” in reference to the composition of the dehydrogenation feed stream, we mean that the linear butenes comprise at least about 95%, at least about 96%, at least about 97%, at least about 98%, at least about 99% of the butenes in the dehydrogenation feed stream.

In a particular embodiment, renewable butadiene is prepared from renewable isobutanol prepared by fermentation as described herein. The isobutanol thus produced is then dehydrated under conditions, as described herein, which maximize the yield of linear butenes (e.g., heterogeneous acidic catalysts such as γ-alumina at about 350° C.). The resulting mixture of ˜1:1 linear butenes/isobutene is then contacted with a dehydrogenation catalyst (e.g., chromium-oxide treated alumina, platinum- and tin-containing zeolites and alumina, cobalt- and molybdenum-containing alumina, etc. at about 450-600° C.) to form a mixture of butadiene and unreacted isobutene. In a specific embodiment, the dehydrocyclization catalyst is a commercial catalyst based on chromium oxide on an alumina support. The isobutene can be isomerized to linear butenes as described herein, and recycled back to the dehydrogenation step in order to produce additional butadiene (thereby increasing the effective yield of butadiene), or used as a raw material for other processes or materials as described herein.

The renewable butadiene thus obtained can then be converted, for example, to a wide variety of renewable polymers and co-polymers by most known methods of polymerization and used in a multitude of commercial applications. As described herein, renewable butadiene can be polymerized or copolymerized with other monomers (which themselves may be renewable monomers or monomers obtained from conventional, non-renewable sources). For example, very low molecular weight polymers and copolymers of butadiene, called telomers or liquid polybutadiene, can be prepared by anionic polymerization using initiators such as n-butyl lithium, often with co-initiators such as potassium tert-butoxide or tert-amines as taught in U.S. Pat. No. 4,331,823 and U.S. Pat. No. 3,356,754. These low molecular weight oligomers (MW 500-3000) can be used in pressure sensitive adhesives and thermosetting rubber applications. Butadiene can also be co- and ter-polymerized with vinyl pyridine and other vinyl monomers (e.g. renewable vinyl monomers) in an emulsion process to form polymers useful in floor polishes, textile chemicals and formulated rubber compositions for automobile tires. Butadiene can also be anionically polymerized with styrene (e.g. renewable styrene) and vinyl pyridine to form triblock polymers as taught in U.S. Pat. No. 3,891,721 useful for films and other rubber applications. Butadiene and styrene can be sequentially, anionically polymerized in non-polar solvents such as hexane, to form diblock and triblock polymers, also called SB elastomers, ranging from rigid plastics with high styrene content to thermoplastic elastomers with high butadiene content. These polymers are useful for transparent molded cups, bottles, impact modifiers for brittle plastics, injection molded toys as well as components in adhesives. Solution polybutadiene can be prepared from butadiene, also by anionic polymerization, using initiators such as n-butyl lithium in non-polar solvents without utilizing a comonomer. These elastomers are non-crosslinked during the polymerization and can be used as impact modifiers in high impact polystyrene and bulk polymerized ABS resins, as well as in adhesives and caulks. Solution polymerized polybutadiene can also be compounded with other elastomers and additives before vulcanization and used in automobile tires. Emulsion (latex) polymerization can also be used to convert butadiene and optionally, other monomers such a styrene, methyl methacrylate, acrylic acid, methacrylic acid, acrylonitrile, and other vinyl monomers, to polymers having both unique chemical structure and designed physical structure suitable for specific end use applications. Emulsion polymerization utilizes water as the continuous phase for the polymerization, surfactants to stabilize the growing, dispersed polymer particles and a compound to generate free radicals to initiate the polymerization. Styrene-butadiene emulsion rubber used for automobile tires can be made by this process. Vinyl acids such as acrylic acid and methacrylic acid can be copolymerized in the styrene butadiene rubber. Low levels (0.5-3%) of vinyl acids improve the stability of the latex and can be beneficial in formulated rubber products such as tires, especially when containing polar fillers. Higher levels of acid in rubber latexes, often called carboxylated latex, are used beneficially in paper coating. Latex polymerization is also used to produce rubber toughened plastics and impact modifiers. Impact modifiers made by latex polymerization are also called core-shell modifies because of the structure that is formed while polymerizing the monomers that comprise the polymer. MBS resins are made by a sequential emulsion process where butadiene (B) and styrene (S) are first polymerized to form the rubber particle core, typically 0.1-0.5 micrometers in diameter, and then methyl methacrylate (M) is polymerized to form a chemically grafted shell on the outer surface of the SB rubber core, for example as taught in U.S. Pat. No. 6,331,580. This impact modifying material is isolated from the latex and blended with plastics to improve their toughness. If acrylonitrile(A) is used in place of the methyl methacrylate, with slight variations in the process, such as disclosed in U.S. Pat. No. 3,509,237 and U.S. Pat. No. 4,385,157, emulsion ABS is the product. ABS is used in injection molding and extrusion processes to produce toys, automobile parts, electronic enclosures and house wares. Nitrile rubber is produced in a similar emulsion polymerization process when butadiene and acrylonitrile are copolymerized together to produce a polar elastomer that is very resistant to solvents. Higher butadiene content in the elastomer provides a softer, more flexible product while higher acrylonitrile content results in more solvent resistance. The rubber is isolated from the latex by coagulation and can be fabricated into gloves, automotive hoses, and gaskets where its high resistance to solvents is an advantage.

Renewable butadiene prepared by the process described herein can also be converted to renewable 1,4-butanediol (BDO) and/or renewable tetrahydrofuran (THF), for example using the process described in JP 10-237017 and JP 2001002600 (illustrated below in Scheme 1), in which butadiene is reacted with acetic acid and oxygen in the presence of a palladium catalyst (liquid phase at about 70° C. and 70 bar, using a promoter such as Sb, Bi, Se or Te) to form 1,4-diacetoxy-2-butene, which is then hydrogenated (liquid phase, at about 50° C. and 50 bar over a conventional hydrogenation catalyst such as Pd/C) to 1,4-diacetoxybutane. Acidic hydrolysis of the 1,4-diacetoxybutane (e.g., using an acidic ion exchange resin) provides BDO and THF in high yield.

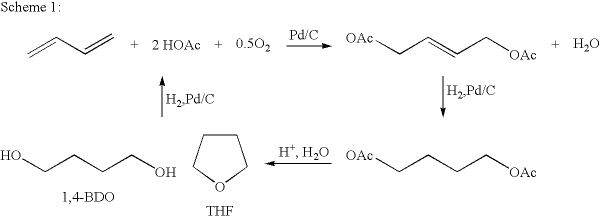

Renewable BDO and THF can be converted to a variety of renewable products. For example renewable BDO can be reacted with a suitable diisocyanates to form renewable Lycra™ and Spandex™ products, as well as thermoplastic urethane elastomers. Renewable BDO can also be used to form renewable polybutylene terephthalate by reacting renewable BDO with terephthalic acid or terephthalate esters, or can be copolymerized with renewable aliphatic diacids such as adipic acid or succinic acid to form renewable aliphatic polyesters such as polybutylene adipate or polybutylene succinate. In some embodiments the terephthalic acid or terephthalate esters can be renewable, prepared by oxidation of renewable xylene made, e.g., by the method described in U.S. Ser. No. 12/327,723 and U.S. 61/295,886. Renewable BDO can also be used to prepare renewable γ-butyrolactone (GBL), renewable pyrrolidone solvents such as N-methylpyrrolidinone (NMP), renewable N-vinylpyrrolidinone (NVP), etc. as illustrated below in Scheme 2:

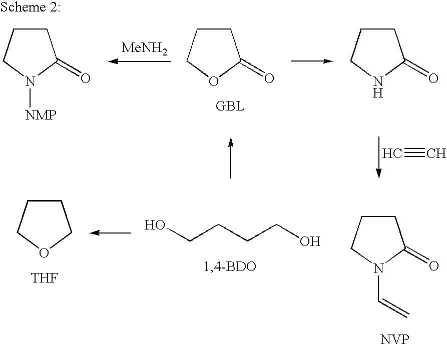

Renewable GBL and NMP can be used as solvents, and renewable NVP can be used in personal care products such as hairspray.

Renewable butadiene prepared by the processes described herein can also be used to form renewable dodecandioic acid (DDDA), or renewable lauryllactam by forming the oxime of the intermediate cyclododecanone, then rearranging the oxime to lauryllactam (e.g., using the method of U.S. Pat. No. 6,649,757). The lauryllactam can then be polymerized to form renewable nylon-12, as shown below in Scheme 3:

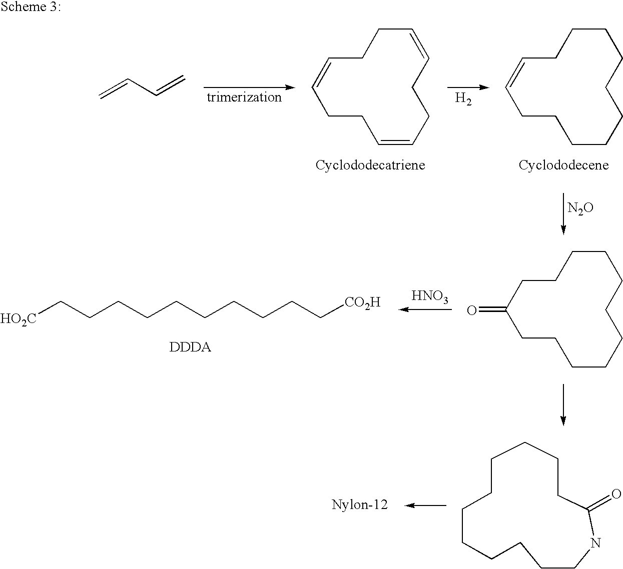

Renewable butadiene prepared by the processes described herein can also be used to prepare renewable chloroprene, which can be polymerized to provide renewable synthetic rubbers. Renewable chloroprene can be prepared by chlorinating renewable butadiene (e.g., free radical, gas phase chlorination with Cl₂ at 250° C. and 1-7 bar to give a mixture of cis and trans-1,4-DCB as well as 3,4-DCB). At butadiene conversions of 10-25%, the selectivity to this mixture of DCBs can be 85-95%. 3,4-dichloro-1-butene (3,4-DCB) can be dehydrochlorinated to form chloroprene (e.g., using dilute alkaline catalysts at 85° C.), as shown below in Scheme 4. The 1,4-DCB by-products can be isomerized to 3,4-DCB using a copper catalyst. In addition, by distilling off the 3,4-DCB during the reaction (b.p. 123° C. vs. 155° C. for the 1,4-isomers), the equilibrium of the reaction can be shifted to provide a selectivity of 95-98%.

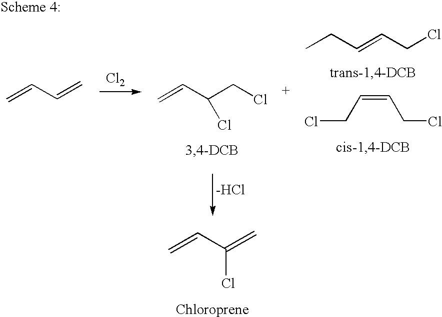

Renewable butadiene prepared by the processes described herein can also be used to prepare renewable nylon-6,6 (Scheme 5). For example, renewable nylon-6,6 can be prepared by reacting renewable butadiene with HCN in the presence of a zero valent nickel catalyst to provide adiponitrile. Adiponitrile can be hydrogenated to form hexamethylenediamine (HMD), and hydrolyzed to form adipic acid. The HMD and adipic acid can then be polymerized to form nylon-6,6.

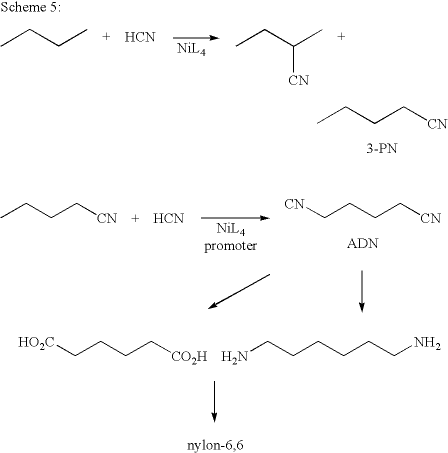

Alternatively, as shown in Scheme 6, renewable adiponitrile can be hydrocyanated and cyclized to renewable caprolactam (CL), e.g., using a doped Raney Ni (using the method of U.S. Pat. No. 5,801,286) and cyclized to CL in the presence of water (using the method of U.S. Pat. No. 5,693,793). The renewable caprolactam can then be polymerized to form renewable nylon-6 using methods known in the art.

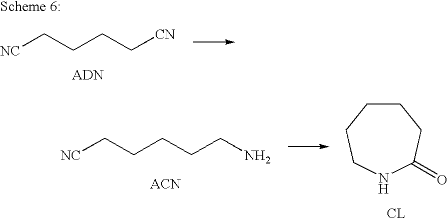

Renewable butadiene prepared by the processes described herein can also be used to prepare renewable sulfolene and sulfolane using the method illustrated in Scheme 7:

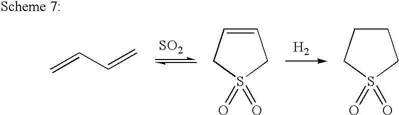

Renewable butadiene prepared by the processes described herein can also be used to prepare renewable styrene, renewable polystyrene, and renewable styrenic polymers (e.g., renewable SBR rubbers). Renewable styrene can be prepared, for example by dimerizing renewable butadiene to form vinylcyclohexene, which can be dehydrogenated in a stepwise fashion to form ethyl benzene (e.g., using the method of WO 2003/070671), then styrene (e.g., using the method of U.S. Pat. No. 4,229,603). Alternatively, vinylcyclohexene can be dehydrogenated directly to styrene. The renewable styrene can be homopolymerized to form renewable polystyrene, copolymerized with renewable butadiene to form SBR rubber, etc.

Renewable butadiene prepared by the processes described herein can also be used to prepare renewable ethylidene norbornene (ENB) for producing completely renewable or partially renewable ethylene-propylene-diene rubber (depending on whether renewable ethylene and/or propylene are used). Renewable ethylene can be prepared by dehydrogenating renewable ethanol (e.g. produced by fermentation or thermochemical methods), and renewable propylene can be prepared, for example by the methods described in U.S. 61/155,029. Renewable ENB can be prepared, for example, by reacting renewable butadiene and dicyclopentadiene in a four-step process. In the first step, dicyclopentadiene is decoupled to cyclopentadiene and reacted with renewable butadiene via Diels-Alder condensation to vinylnorbornene (VNB). This is followed by distillation to obtain refined VNB, which is catalytically isomerized (U.S. Pat. No. 4,720,601) to ENB.

Renewable butadiene prepared by the processes described herein can also be thermally dimerized to form renewable 1,5-cyclooctadiene (COD) using the methods of, e.g., U.S. Pat. No. 4,396,787. Renewable COD can be used in the preparation of renewable ethylene oligomerization catalysts such as Ni(COD)₂. Butadiene can also be dimerized to produce 1-octene and 1-octanol.

In other embodiments, the dehydration of 3-methyl-1-butanol produces a mixture of methyl butenes and small amounts of other pentenes which upon treatment with a dehydrogenation catalyst forms primarily isoprene from methylpentenes (e.g. 2-methyl-1-butene, 2-methyl-2-butene, 3-methyl-1-butene), for example 3-methyl-1-butene, and other pentadienes, such as 1,3-pentadiene, from other pentenes. The pentadienes are separated from each other by distillation. Dehydration catalysts and conditions are optimized to produce varying amounts of specific olefins, and their resulting di-olefins upon treatment with a dehydrogenation catalyst.

The purification of isobutene as described above produces renewable isobutene that meets all current industrial specifications and can be used to manufacture all chemicals and materials currently produced e.g., from conventional petroleum-based isobutene. For example, renewable or partially renewable polyisobutylene, butyl rubber, methyl methacrylate, isoprene, and other chemicals can be produced by the methods of the present invention. Renewable isobutene can also be oxidized under suitable conditions to provide methacrylic acid and methacrylic acid esters (Scheme 8). Isobutene can be oxidized over suitable metal oxide catalysts (e.g., using the methods described in JP 2005-253415) at temperatures of about 300-500° C. to methacrolein (MAL) which is then further oxidized to methacrylic acid (MMA) (WO 2003053570) at temperatures of about 350-500° C. The resultant methacrylic acid can be further esterified to methylmethacrylate. The oxidation of isobutene to MMA may also be accomplished in a single step (e.g. as described in WO2003053570).

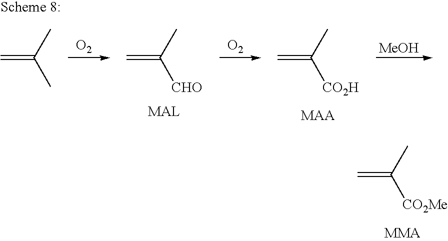

An alternative process for the preparation of MMA is by the oxidative esterification of MAL to MMA (e.g., as described in U.S. Pat. No. 4,518,796) using catalysts such as Pd/Pb/Mg—Al₂O₃ (e.g., as described in JP 2006306731) and Pd₅Bi₂Fe/CaCO₃ (Scheme 9.

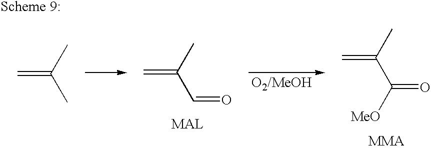

Additionally, all materials currently produced from butadiene such as synthetic rubbers and nylon can be manufactured from the renewable butadiene produced by the dehydrogenation of renewable butenes according to the present invention. For example, butadiene is used directly as a monomer and co-monomer for the production of synthetic rubber. It is also converted into “oxidized” monomers such as 1,4-butanediol, adiponitrile, and adipic acid as described herein for the production of polyester and nylon materials (e.g., adipic acid is produced by the hydrocarboxylation of butadiene in the presence of a suitable catalyst, CO and water; e.g., adiponitrile is produced by the hydrocyanation of butadiene in the presence of a suitable catalyst). The production of renewable isoprene from the dehydrogenation of methylbutenes or the hydroformylation and dehydration of renewable isobutene allows the preparation of renewable or partially renewable versions of all chemicals and materials produced from isoprene, especially synthetic rubber and other polymers.

One of the major industrial uses of isobutene is in the production of butyl rubber primarily for use in automobile tires. Butyl rubber is a high performance polymer comprised of high purity isobutene crosslinked with di-olefins such as butadiene or isoprene (e.g. U.S. Pat. No. 2,984,644; Dhaliwal G K, Rubber Chemistry and Technology 1994, 67, p. 567). Typically, 1-3% of isoprene is blended with isobutene and co-polymerized in the presence of a polymerization catalyst such as aluminum chloride and other metal salts.

In some embodiments, renewable isoprene is produced by contacting 3-methyl-1-butanol or 2-methyl-1-butanol with a dehydration catalyst and a dehydrogenation catalyst, under conditions similar to those described herein for preparing renewable butadiene. The renewable isoprene thus formed is then blended with renewable isobutene, obtained by the methods described above or by conventional methods such as hydration of isobutylene to t-butanol and subsequent dehydration to isobutene, to form a renewable monomer feedstock for the production of renewable butyl rubber. Petroleum-based isoprene and isobutene can also used with the renewable isoprene and/or isobutene to produce butyl rubber that is partially renewable. In addition to blending purified isoprene with purified isobutene to produce butyl rubber, a renewable blend of isobutene and isoprene can be produced by contacting a mixture of isobutanol and 3-methyl-1-butanol (or 2-methyl-1-butanol) with a dehydration catalyst to form isobutylene and 3-methyl-butenes (or 2-methyl-butenes) and then contacting this olefin mixture with a dehydrogenation catalyst to form isobutene and isoprene. By-products such as butadiene and other C₅ olefins and di-olefins are removed by extractive distillation to give mixtures containing only isobutene and isoprene. The amount of isoprene in the mixture can be controlled by manipulating the 3-methyl-1-butanol producing pathway in the host microorganism or the appropriate selection of catalyst in the thermochemical conversion of biomass. In some embodiments, the 3-methyl-1-butanol (or 2-methyl-1-butanol) concentration is tuned to 1-3% of the isobutanol produced such that the resulting isobutene/isoprene mixture can be directly used to produce butyl rubber. Alternatively, in other embodiments a higher concentration of 3-methyl-1-butanol is produced to form a mixture of isobutene and isoprene that is then diluted with pure isobutene to optimize butyl rubber production. The isoprene produced from 3-methyl-1-butanol (or 2-methyl-1-butanol) containing isobutanol is also separately removed and blended with isobutene to the appropriate concentration. Alternatively, the butadiene produced by the dehydrogenation of 1- and 2-butenes is used as a cross-linking agent in a butyl rubber product.

### Example 1

A cellulosic material consisting of 45% cellulose, 25% hemicellulose, 22% lignin and 8% other materials is pretreated to yield a slurry of 8% insoluble cellulose with about 4% insoluble lignin, 1% glucose, 40 g/L xylose, 2 g/L mannose, 2 g/L galactose, 1 g/L arabinose, 5 g/L acetic acid in solution. The slurry is fed into an agitated saccharification and fermentation vessel and charged with cellulase enzyme sufficient to hydrolyze 80% of the cellulose 72 hours. A microorganism known to ferment glucose, xylose, mannose, galactose and arabinose to isobutanol is added to the fermentation, and the vessel is agitated for 72 hours. Isobutanol produced by the fermentation is separated from the fermentation broth by distillation. The first isobutanol-containing distillation cut contains 20% w/w isobutanol and 80% w/w water that condenses to form two phases—a light phase containing 85% isobutanol and 15% water and a heavy phase containing 8% isobutanol and 92% water. The light phase is distilled a second time and two low-water cuts of isobutanol are obtained. One cut is comprised of 99.5% isobutanol and 0.5% water while the second cut is comprised of 98.8% isobutanol, 1% 3-methyl-1-butanol, and 0.2% water.

### Example 2

Isobutanol obtained in Example 1 was fed through a preheater and to a fixed-bed tubular reactor packed with a commercial dehydration catalyst (BASF AL3996). The internal reactor temperature was maintained at 300° C. and the reactor pressure was atmospheric. The WHSV of the isobutanol was 6 hr⁻¹. Primarily isobutene and water were produced in the reactor and separated in a gas-liquid separator at 20° C.; the water had 1% of unreacted isobutanol and conversion was 99.8%. GC-MS of the gas phase effluent indicated it was 96% isobutene, 2.5% 2-butene (cis and trans) and 1.5% 1-butene.

### Example 3

Isobutanol obtained in Example 1 is fed through a preheater and to a fixed-bed tubular reactor packed with a commercial dehydration catalyst (e.g., an X-type zeolite). The internal reactor temperature is maintained at 370° C. and the reactor pressure is atmospheric. The WHSV of the isobutanol is 3 hr⁻¹. A mixture of C₄ olefins and water are produced in the reactor and separated in a gas-liquid separator at 20° C.; the water has <1% of unreacted isobutanol and conversion is >99.8%. GC-MS of the gas phase effluent indicates it is 50% isobutene, 40% 2-butene (cis and trans) and 10% 1-butene.

### Example 4

A mixture of 50% 2-methyl-1-butanol and 50% 3-methyl-1-butanol (v/v) is fed through a preheater and to a fixed-bed tubular reactor packed with a commercial dehydration catalyst (e.g., BASF AL3996). The internal reactor temperature is maintained at 400° C. and the reactor pressure is atmospheric. The WHSV of the alcohol feed is 2 hr⁻¹. A mixture of C₅ olefins and water are produced in the reactor and separated in a gas-liquid separator at 50° C. A two phase liquid is obtained which is approximately 50% unreacted C₅ alcohols and 50% water indicating a total conversion of 90%. GC-MS of the gas phase effluent indicates it is 40% 2-methyl-1-butene, 30% 3-methyl-1-butene, and 30% 2-methyl-2-butene.

### Example 5

A mixture of 99% Isobutanol and 1% 3-methyl-1-butanol is fed through a preheater and to a fixed-bed tubular reactor packed with a commercial dehydration catalyst (e.g., BASF AL3992). The internal reactor temperature is maintained at 350° C. and the reactor pressure is atmospheric. The WHSV of the isobutanol mixture is 5 hr⁻¹. A mixture of C₄ olefins, C₅ olefins, and water are produced in the reactor and separated in a gas-liquid separator at 50° C.; the water has <1% of unreacted isobutanol and trace 3-methyl-1-butanol indicating conversion of >99.8%. GC-MS of the gas phase effluent indicates it is 70% isobutene, 20% 2-butene (cis and trans), 9% 1-butene, 0.7% 3-methyl-1-butene, and 0.3% 2-methyl-2-butene.

### Example 6

1-butanol is fed through a preheater and to a fixed-bed tubular reactor packed with a commercial dehydration catalyst (e.g., BASF AL3996). The internal reactor temperature is maintained at 370° C. and the reactor pressure is atmospheric. The WHSV of the 1-butanol is 2 hr⁻¹. A mixture of C₄ olefins and water are produced in the reactor and separated in a gas-liquid separator at 20° C. The water has 5% 1-butanol indicating a total conversion of 99%. GC-MS of the gas phase effluent indicates it is 40% 2-butene (cis and trans), 35% 1-butene, and 25% isobutene.

### Example 7

2-butanol is fed through a preheater and to a fixed-bed tubular reactor packed with a commercial dehydration catalyst (e.g., BASF AL3996). The internal reactor temperature is maintained at 350° C. and the reactor pressure is atmospheric. The WHSV of the 2-butanol is 2 hr⁻¹. A mixture of C₄ olefins and water are produced in the reactor and separated in a gas-liquid separator at 20° C. The water has 2.5% 2-butanol indicating a total conversion of 99.5%. GC-MS of the gas phase effluent indicates it is 50% 2-butene (cis and trans), 30% 1-butene, and 20% isobutene.

### Example 8

A mixed butene stream from Example 2, containing 96% isobutene, 2.5% 2-butenes (cis and trans), and 1.5% 1-butene is mixed with air at a relative feed rate of 10:1 butenes:air. The resultant mixture is 1.9% oxygen and 3.6% linear butenes. The mixture is preheated to 400° C. and fed at a GHSV of 300 hr⁻¹ to a fixed-bed tubular reactor loaded with 2 catalyst beds in sequence; the first contains ZnFe₂O₄ and the second contains CO₉Fe₃BiMoO₅₁. The effluent from the reactor is dried over a molecular sieve column to remove water. Nitrogen and oxygen are removed by passing the C₄ stream through a gas-liquid separator at −78° C. (dry ice bath). The C₄ product is analyzed via GC-MS. The composition is found to be 96% isobutene, 3.9% butadiene, and 0.1% linear butenes. butadiene is stripped from the gas stream by extraction with acetonitrile. The resultant stream is 99.9% isobutene and 0.1% linear butenes with trace butadiene (<0.01%).

### Example 9

A mixed butene stream from Example 3, containing 50% isobutene, 40% 2-butenes (cis and trans), and 10% 1-butene is mixed with air at a relative feed rate of 4:5 butenes:air. The resultant mixture is 11.7% oxygen and 22.2% linear butenes. The mixture is preheated to 400° C. and fed at a GHSV of 300 hr⁻¹ to a fixed-bed tubular reactor loaded with 2 catalyst beds in sequence; the first contains ZnFe₂O₄ and the second contains CO₉Fe₃BiMoO₅₁. The effluent from the reactor is dried over a molecular sieve column to remove water. Nitrogen and oxygen are removed by passing the C₄ stream through a gas-liquid separator at −78° C. (dry ice bath). The C₄ product is analyzed via GC-MS. The composition is found to be 50% isobutene, 49.9% butadiene, and 0.1% linear butenes. butadiene is stripped from the gas stream by extraction with acetonitrile. The resultant stream is 99.9% isobutene and 0.1% linear butenes with trace butadiene (<0.01%).

### Example 10

A stream containing 70% isobutene, 20% 2-butene (cis and trans), 9% 1-butene, 0.7% 3-methyl-1-butene, and 0.3% 2-methyl-2-butene from Example 5 is mixed with air at a relative feed rate of 4:3 olefin:air. The resultant mixture is 9% oxygen and 17.1% linear butenes+C₅ olefins. The mixture is preheated to 400° C. and fed at a GHSV of 300 hr⁻¹ to a fixed-bed tubular reactor loaded with 2 catalyst beds in sequence; the first contains ZnFe₂O₄ and the second contains CO₉Fe₃BiMoO₅₁. The effluent from the reactor is dried to remove water. Nitrogen and oxygen are removed by passing the C₄ stream through a gas-liquid separator at −78° C. (dry ice bath). The hydrocarbon product is analyzed via GC-MS. The composition is found to be 70% isobutene, 28.9% butadiene, 0.1% linear butenes, and 1% isoprene. butadiene and isoprene are stripped from the gas stream by extraction with acetonitrile. The resultant stream is 99.9% isobutene and 0.1% linear butenes with trace butadiene (<0.01%). Isoprene and butadiene are separated by distillation to produce purified butadiene and isoprene.

### Example 11

120 sccm of nitrogen and 120 sccm of 2-butene (mixture of cis and trans) was fed through a preheater and to a fixed-bed tubular reactor packed with 15 g of a commercial Cr₂O₃ on alumina dehydrogenation catalyst (BASF Snap catalyst). The internal reactor temperature was maintained at 600° C. and the reactor pressure was atmospheric. The WHSV of the 2-butene was about 1 hr⁻¹. GC-FID of the gas phase effluent indicated it was 74% linear butenes (mixture of 1-, cis-2-, and trans-2-), 16% butadiene, 2.5% n-butane, and 7.5% C₁-C₃ hydrocarbons. The resulting conversion of 2-butene was 26% (ignoring rearrangement to 1-butene) with a selectivity to butadiene of 61.5% based on % carbon.

### Example 12

120 sccm of nitrogen and 120 sccm of isobutylene was fed through a preheater and to a fixed-bed tubular reactor packed with 15 g of a commercial Cr₂O₃ on alumina dehydrogenation catalyst (BASF Snap catalyst). The internal reactor temperature was maintained at 600° C. and the reactor pressure was atmospheric. The WHSV of the isobutylene was about 1 hr⁻¹. GC-FID of the gas phase effluent indicated it was 78.8% isobutylene, 13.6% isobutane, and 7.6% C₁-C₃ hydrocarbons. No butadiene was produced from the isobutylene.

### Example 13

Renewable wet isobutanol (containing 15% water and ˜4% ethanol) obtained from fermentation was fed through a preheater and to a fixed-bed tubular reactor packed with a commercial γ-alumina dehydration catalyst (BASF Snap catalyst). The internal reactor temperature was maintained at 400° C. and the reactor pressure was atmospheric. The WHSV of the isobutanol was ˜0.1 hr⁻¹. The products were separated in a gas-liquid separator at 20° C., where relatively pure water was removed as the liquid product. The gas phase product was dried over a molecular sieve bed. GC-FID of the gas phase effluent from the dehydration reactor was 82% isobutylene, 13% linear butenes (mixture of 1-butene, and cis- and trans-2-butene), 4.5% ethylene, and 0.5% propylene. The flow of the gas-phase stream was ˜120 sccm. This stream was combined with 120 sccm of nitrogen and was fed through a preheater and to a fixed-bed tubular reactor packed with 15 g of a commercial Cr₂O₃ on alumina dehydrogenation catalyst. The internal reactor temperature was maintained at 600° C. and the reactor pressure was atmospheric. The WHSV of the mixed butene stream was about 1 hr⁻¹. GC-FID of the gas phase effluent indicated it was 78.5% isobutylene with 2.5% isobutane, 7.5% linear butenes, 3.7% ethylene with 0.6% ethane, 2.9% butadiene, and the remaining 4.4% was methane and propylene. This indicates an approximate yield of 22% butadiene based on linear butenes fed to the dehydrogenation reactor.

1. A method of preparing butadiene comprising:
(a) providing an alcohol mixture comprising one or more butanols;
(b) contacting said alcohol mixture with a dehydration catalyst, thereby forming an olefin mixture comprising one or more linear butenes and isobutene;
(c) contacting the olefin mixture of (b) with a dehydrogenation catalyst, thereby forming a di-olefin mixture comprising butadiene and isobutene; and
(d) isolating butadiene from the di-olefin mixture of (c).

2. The method of claim 1, wherein the alcohol mixture comprises one or more renewable butanols.

3. The method of claim 2, wherein the alcohol mixture comprises renewable isobutanol.

4. The method of claim 2, wherein the one or more renewable butanols are prepared by fermentation.

5. The method of claim 4, wherein the fermentation comprises fermenting with a genetically modified microorganism.

6. The method of claim 2, wherein the one or more renewable butanols are prepared by hydrogenation of one or more butyric acids produced by anaerobic digestion of biomass.

7. The method of claim 1, wherein the olefin mixture of (b) comprises at least about 10% linear butenes.

8. The method of claim 1, wherein prior to step (c), isobutene is substantially removed from the olefin mixture.

9. The method of claim 1, wherein said dehydrogenation is carried out in the presence of an inert carrier gas, or carried out at a pressure of about 0.1 atm to about 0.7 atm.

10. The method of claim 1, wherein said dehydrogenation is carried out in the presence of oxygen.

11. The method of claim 1, wherein said isolating comprises extractive distillation.

12. A method of preparing isoprene comprising:
(a) providing an olefin mixture comprising one or more pentenes, with the proviso that at least a portion of the olefin mixture comprises one or more methylbutenes;
(b) contacting the olefin mixture of (a) with a dehydrogenation catalyst, thereby forming a mixture comprising isoprene; and
(c) isolating isoprene from the mixture of step (b).

13. The method of claim 12, wherein said providing an olefin mixture of step (a) comprises:
(a1) providing an alcohol mixture comprising one or more pentanols; and
(a2) contacting said alcohol mixture with a dehydration catalyst, thereby forming the olefin mixture.

14. The method of claim 13, wherein the olefin mixture of (a2) comprises at least about 50% methylbutenes.

15. The method of claim 13, wherein the mixture of step (b) comprises at least about 50% isoprene.

16. The method of claim 13, wherein the alcohol mixture comprises renewable alcohols.

17. The method of claim 16, wherein the renewable alcohols are prepared by fermentation.

18. The method of claim 17, wherein the fermentation comprises fermenting with a genetically modified microorganism.

19. The method of claim 13, wherein alcohol mixture comprises 3-methyl-1-butanol or 2-methyl-1-butanol.

20. The method of claim 13, wherein the alcohol mixture comprises 3-methyl-1-butanol.

21. The method of claim 13, wherein the alcohol mixture comprises 2-methyl-1-butanol.

22. The method of claim 12, wherein said isolating comprises extractive distillation.

23. The method of claim 12, wherein said dehydrogenation is carried out in the presence of an inert carrier gas, or carried out at a pressure of about 0.1 atm to about 0.7 atm.

24. The method of claim 11, wherein said dehydrogenation is carried out in the presence of oxygen.

25. A method of preparing isobutene comprising:
(a) providing an olefin mixture comprising one or more linear butenes and isobutene;
(b) contacting the olefin mixture of (a) with a dehydrogenation catalyst, thereby forming a di-olefin mixture comprising butadiene and isobutene; and
(c) isolating isobutene from the mixture of (b).

26. The method of claim 25, wherein said providing an olefin mixture of step (a) comprises:
(a1) providing an alcohol mixture comprising one or more butanols; and
(a2) contacting said alcohol mixture with a dehydration catalyst, thereby forming the olefin mixture.

27. A method of preparing a renewable monomer, comprising:
(a) providing an alcohol mixture comprising one or more renewable butanols;
(b) contacting said alcohol mixture with a dehydration catalyst, thereby forming an olefin mixture comprising one or more renewable linear butenes and renewable isobutane;
(c) removing at least a portion of the renewable isobutene from the olefin mixture, thereby forming an isobutene depleted olefin mixture;
(d) contacting the isobutene depleted olefin mixture of (c) with a dehydrogenation catalyst, thereby forming a dehydrogenation mixture comprising renewable butadiene;
(e) isolating recovering renewable butadiene from the dehydrogenation mixture of step (d); and
(f) converting the renewable butadiene to a renewable monomer selected from the group consisting of 1,4-butanediol, THF, N-vinylpyrrolidinone, lauryllactam, chloroprene, adipic acid, hexamethylenediamine, caprolactam, and ethylidene norbornene.

28. Renewable butadiene prepared by the method of claim 1.

29. Renewable isoprene prepared by the method of claim 12.

30. Renewable isobutene prepared by the method of claim 25.

31. A renewable monomer prepared by the method of claim 27, wherein the monomer is selected from the group consisting of 1,4-butanediol, THF, N-vinylpyrrolidinone, lauryllactam, chloroprene, adipic acid, hexamethylenediamine, caprolactam, and ethylidene norbornene.

32. A renewable polymer prepared by polymerizing or copolymerizing the renewable butadiene of claim 28.

33. A renewable polymer prepared by the polymerization or copolymerization of the renewable isoprene of claim 29.

34. A renewable polymer prepare by polymerizing or copolymerizing the renewable isobutene of claim 30.

35. A renewable polymer prepared by the polymerization or copolymerization of one or more of the renewable monomers of claim 31.

36. The renewable polymer of claim 32, selected from the group consisting of, liquid polybutadienes, SB elastomers, MBS resins, ABS resins, and nitrile rubbers.

37. The renewable polymer of claim 33, selected from the group consisting of polyisoprene, styrene-isoprene block copolymers, and isoprene-containing butyl rubber.

38. The renewable polymer of claim 34, selected from the group consisting of polyisobutylenes and butyl rubbers.

39. The renewable polymer of claim 35, wherein the polymer is selected from the group consisting of polyesters, nylons, nylon-12, nylon-6,6, nylon-6, polyisocyanates, polychloroprenes, polystyrenes, SBR rubbers, ethylene-propylene-diene rubbers, and polymethylmethacrylates.

40. The method of claim 1, wherein at least a portion of the isobutene of the olefin mixture produced in step (b) is rearranged to an isomer mixture comprising one or more linear butenes, and said isomer mixture and the one or more linear butenes of the olefin mixture are combined prior to said contacting with the dehydrogenation catalyst in step (c).

41. The method of claim 1, wherein the dehydrogenation catalyst of step (c) also catalyzes rearrangement of at least a portion of the isobutene of the olefin mixture, whereby after contacting the dehydrogenation catalyst, at least a portion of the isobutene rearranges to one or more linear butenes, at least a portion of which dehydrogenate to butadiene.

42. The method of claim 2, wherein the one or more renewable butanols are prepared photosynthetically or thermochemically.

43. A method of preparing isoprene comprising:
(a) providing an olefin mixture comprising one or more linear butenes and isobutene;
(b) contacting the olefin mixture of (a) with a dehydrogenation catalyst, thereby forming a di-olefin mixture comprising butadiene and isobutene; and
(c) isolating isobutene from the mixture of (b);
(d) reacting the isobutene with formaldehyde in the presence of an acidic catalyst, thereby forming isoprene.
